(19) United States
(12) Patent Application Publication
(10) Pub. No.: US 2008/0249159 A1
(43) Pub. Date: Oct. 9, 2008

(54) Method of using oral lipase inhibitors to reduce plasma ghrelin and to prevent weight regain

(76) Inventor: Ronald J. Thompson, Ft. Thomas, KY (US)

Correspondence Address: Don Halgren, 35 Central Street, Manchester, MA, 01944, US

(21) Appl. No.: 12/070,069

(22) Filed: Feb. 14, 2008

Related U.S. Application Data
(63) Continuation of application No. 11/602,935, filed on Nov. 21, 2006, now abandoned.

(51) Int. Cl. A61K 31/365; A61P 3/00

(52) U.S. Cl. 514/449

(57) ABSTRACT

A method of reducing ghrelin levels in a human body for weight reduction and to permanently reduce appetite and eliminate weight regain by the step of orally ingesting tetrahydrolipstatin—THL (Orlistat) on a regular basis for a period of at least one to two years.

### FIELD OF THE INVENTION

The present invention relates to the oral use of lipase inhibitors to reduce plasma ghrelin levels in a being, to prevent weight regain by that being, and is a continuation application of co-pending U.S. patent application Ser. No. 11/602,936, filed Nov. 21, 2006, and incorporated herein by reference in its entirety.

### BACKGROUND OF THE INVENTION AND PRIOR ART

### Lipase Inhibitors

Ingested fats are long chain fatty acids that cannot be absorbed in the gastrointestinal system. The presence of food in the stomach signals the release of the lipase enzyme from the pancreas. The lipase enzyme breaks down the long chain fatty acids into short chain fats. The short chain fats are then absorbed from the small intestines into the blood stream. Lipase inhibitors such as orlistat (XENICAL®) (tetrahydrolipstatin—THL) bind to the lipase enzyme and prevent the breakdown of long chain fatty acids. Therefore the fats are not absorbed and passed through the gastrointestinal system as long chain fatty acids.

### Ghrelin

Ghrelin is a recently discovered endocrine hormone produced in the stomach and released into the circulatory blood. Ghrelin, in the circulating blood, stimulates Ghrelin-specified receptors in the brain (hypothalamus) that creates a sensation of hunger. Conversely, a reduced ghrelin level in blood level creates the sensation of satiety, therefore creates appetite reduction.

U.S. Pat. No. 4,598,089 filed Jan. 18, 1984 and issued Jul. 1, 1986, defines tetrahydrolipstatin and its unique gastrointestinal lipase inhibitor actions. These actions are further defined in patents 5,245,056 and 5,399,720, to treat obesity and various medical conditions associated with obesity, specifically diabetes and hypertension. U.S. Pat. No. 6,696,467 further teaches and defines the specific benefits of the lipase inhibitor, THL, for the treatment of obesity by weight reduction and appetite suppression. U.S. Pat. No. 6,004,996 describes the production of THL into microspheres for optimal therapeutic delivery into the lumen of the stomach. Each of these patents are incorporated herein by reference in their entirety.

Ghrelin was only recently discovered, therefore, there are only limited U.S. patents that even describe methods to reduce circulating ghrelin and create satiety, or appetite suppression. U.S. Pat. No. 6,967,237, filed Nov. 15, 2002 (provisional application date of May 30, 2000) and issued Nov. 22, 2005, describes a ghrelin analog to block the action of circulating ghrelin at the hypothalamus. U.S. Pat. No. 6,675,809, filed Aug. 27, 2001 and issued Jan. 13, 2004, defines an intra-luminal (stomach) expandable device to produce satiety by mechanically interfering with ghrelin production/release. U.S. Patent application 20060025808 filed Jan. 22, 2005 and published Feb. 3, 2006, describes a device to bulk the organ's vagal nerve trunks and prevent normal production and release of ghrelin by the ghrelin cells in the stomach, by altering normal sympathetic innervation and neurologic stimulus. U.S. patent application 20040107130 entitled “Physiologic Gastric Impass by Appetite Suppression” filed Jul. 29, 2003 and published Jan. 3, 2004, describes a method to create atrophy of the ghrelin cells and therefore produces long-term appetite suppression and satiety, even after discontinuation of therapy. Each of these patents/applications are also incorporated herein by reference in their entirety.

### Endocrine Verses Exocrine

Endocrine hormones like thyroid, insulin, estrogen, testosterone, progesterone and ghrelin are produced in specific endocrine cells. These hormone-specific cells produce a single type of hormone. The hormone is released into the circulating blood and therefore available to all cells of the body, but only exerts an effect on tissues that are target-specific for that hormone. The ghrelin-specific target tissue is the (brain) hypothalamus. In the hypothalamus, a high concentration of ghrelin stimulates hunger and appetite. Conversely, a low concentration of ghrelin is perceived as satiety, or the lack of hunger and appetite.

Exocrine, or secretory cells, line the digestive and respiratory systems. In the digestive tract, these secretory cells release their secretions into the lumen of the hollow viscus to admix with food to accomplish digestion. After digestion of food occurs, the blood vessels absorb the nutrients and circulate these nutrients, including short chain fats and carbohydrates to all cells of the body.

The stomach as well as part of the small intestines contain both endocrine and exocrine cells. Exocrine cells produce digestive enzymes. Stomach endocrine cells produce ghrelin, cholecystokenin, PPY, pancreatic peptide (PP) and other hormones that help in digestion of food. Cholecystokenin for example, is produced in stomach wall specified endocrine cells, released into the circulating blood, and has a targeted effect on the gallbladder to contract and therefore release the bile stored in the gallbladder into the stomach. The bile aids in digestion of fats, and a high fat content meal is associated with an abundant release of cholecystokenin and bile. Ghrelin production, stimulation of ghrelin release and down regulation are not yet well understood by medical science.

Dr. K L Gettinn, in the article entitled “Effect of fatty acid chain length on suppression of ghrelin and stimulation of PYY, GLP-2 and PP Secretion in Healthy Men,” published in the July 2006 journal PEPTIDES, states “We conclude that the effects of intraduodenal fatty acids on ghrelin, PYY and GLP2 secretion are dependent on their chain length.” Each article cited hereinabove incorporated herein by reference.

Therefore, the increased percentage of long chain fatty acids reduces the serum ghrelin level, and since Orlistat increases the percentage of long chain fatty acids, Orlistat can be expected to reduce the serum ghrelin. With continuous “long-term” use of Orlistat, the endocrine cells that produce ghrelin in the stomach and small intestines can be now, under the current invention, be expected to atrophy. With atrophied ghrelin cells, appetite would be decreased because of the decreased serum ghrelin. With long-term Orlistat use, the atrophied ghrelin cells, decreased appetite because of the decreased ability to produce ghrelin post orlistat therapy, all would translate to decreased weight regain after such Orlistat therapy for at least about two years. Indeed, the “package insert” documents the lack of weight regain after Orlistat therapy, but fails to recognize, anticipate, expect or appreciate the present invention which requires the need to stay on the Orlistat therapy for the long term, which in this invention is at least about two years, so as to permanently atrophy the endocrine cells which would otherwise generate more ghrelin!

XENICAL® (2005 Physician's Desk Reference) Effect on Weight Regain, and incorporated herein by reference, recites as follows:

“In study 14119C patients treated with placebo regained 52% of the weight they had previously lost while the patients treated with XENICAL® regained 26% of the weight they had lost (p<0.001).”

“In study 14185, patients treated with placebo regained 63% of the weight they had previously lost while patients treated with XENICAL® regained 35% of the weight they had lost (p<0.001).”

“In study 14302, patients treated with placebo regained 53% of the weight they had previously lost while the patients treated with XENICAL ® regained 32% of the weight that they had lost (p<0.001).”

Serum ghrelin determinations were not preformed in any of the XENICAL® weight regain studies referenced above, which cited studies are incorporated herein by reference. Ghrelin was not even discovered until several years after these studies were published. Therefore, we inventively stipulate that the “mechanism of action” of the long term use (at least about two years) of Orlistat for associated prevention of weight regain is the actual “atrophy of the ghrelin-producing cells”.

### DESCRIPTION OF THE DRAWINGS

The objects and advantages of the present invention will become more apparent when viewed in conjunction with the following drawings in which:

FIG. 1 represents a cross-sectional view of a normal human stomach before any Orlistat therapy;

FIG. 2 represents a cross-sectional view of that normal human stomach after six months of Orlistat therapy, displaying a shrinkage thereof;

FIG. 3 represents a cross-sectional view of that normal human stomach after twelve months of Orlistat therapy, displaying a further shrinkage thereof;

FIG. 4 represents a cross-sectional view of normal human stomach in a “pre-prandial” state, with a circle placed thereon to designate a subsequently viewed enlarged portion thereof;

FIG. 5 represents the enlarged portion shown within the drawing of FIG. 4, with large plump ghrelin cells, showing abundant ghrelin hormone production released into the bloodstream from ghrelin cells, the ghrelin circulating towards the hydrothalmus to stimulate an appetite;

FIG. 6 represents a cross-sectional view of a pre-prandial stomach after six months of Orlistat therapy, with a circle placed thereon to designate a subsequently viewed enlarged portion thereof;

FIG. 7 represents the enlarged portion in the circle shown in FIG. 6 showing the grhelin cells smaller and partially atrophied, with some ghrelin cells completely atrophic, wherein they can not produce ghrelin, with much less ghrelin hormones shown in the blood vessels because of the atrophy of those ghrelin producing cells;

FIG. 8 represents a cross-sectional view of a pre-prandial stomach shown after twelve months of Orlistat therapy, and the Orlistat therapy has been discontinued after such a minimum portion of the inventive period, with a circle placed thereon to designate a subsequently viewed enlarged portion thereof; and

FIG. 9 represents the enlarged portion of FIG. 7 within the circle, showing ghrelin cells with some atrophied to a very small state, and most ghrelin cells completely atrophic which cannot now produce ghrelin, and showing very little ghrelin hormones circulating in the bloodstream, thereby decreasing appetite, all of which comprises the etiology of reduced weight regain after such inventive prolonged Orlistat therapy.

### DESCRIPTION OF THE PRESENT INVENTION

The present invention comprises the use of long-term orlistat therapy (of several years duration) to: depress the serum ghrelin level by presenting long chain fatty acids to the stomach and duodenum; induce ghrelin cell atrophy; reduce post-Orlistat therapy ghrelin production, to reduce post-orlistat ghrelin associated appetite so as to prevent post Orlistat weight regain.

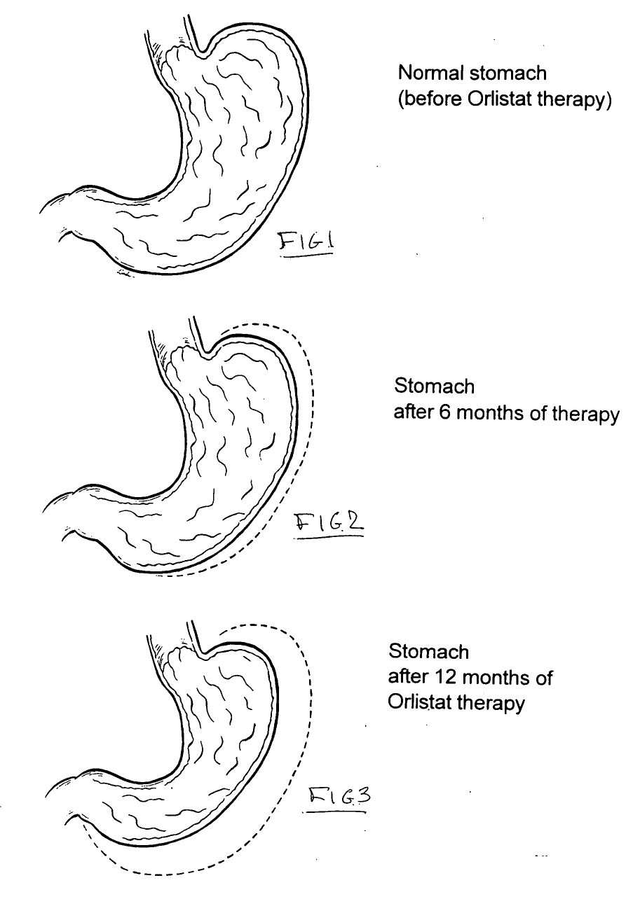

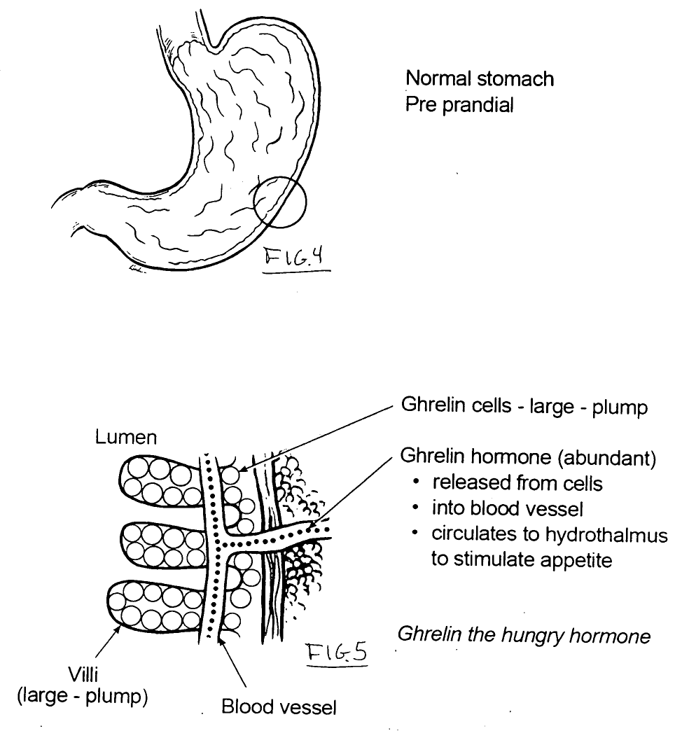

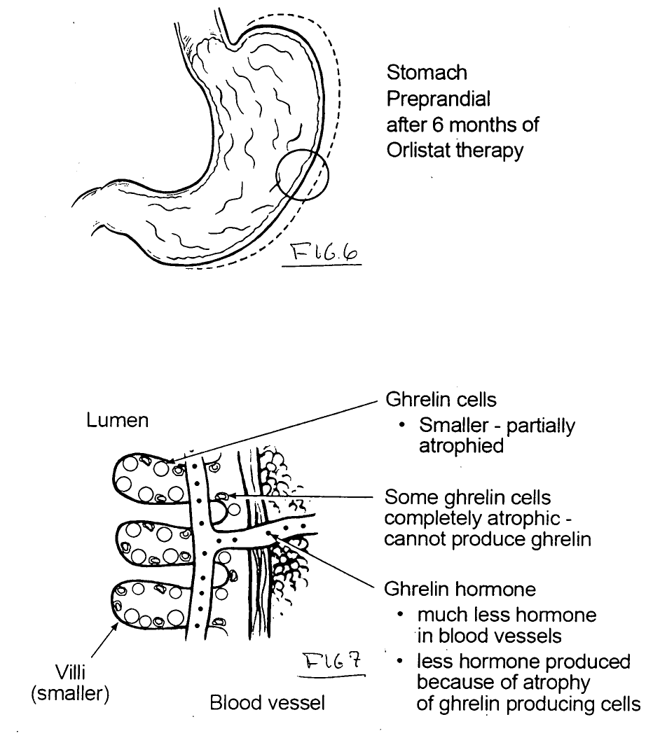

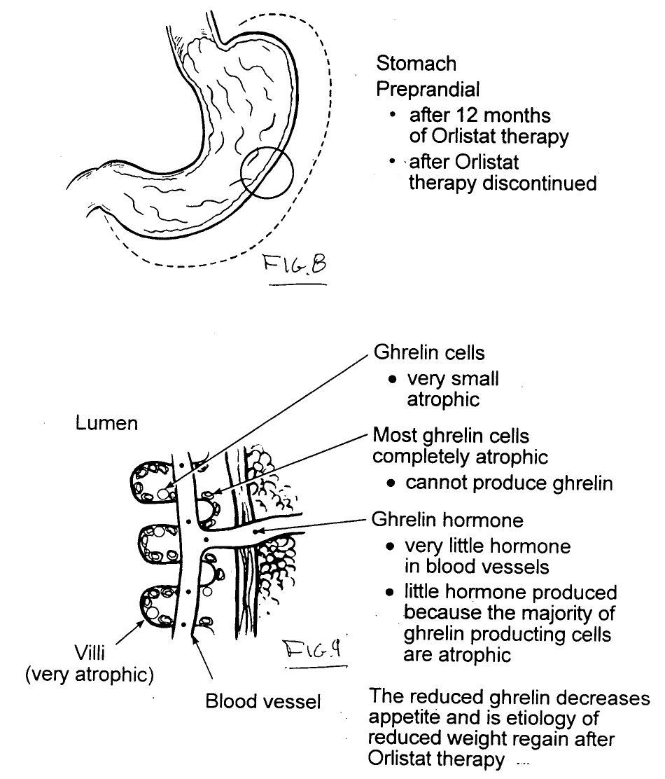

The present invention thus comprises a method of depressing serum ghrelin levels in a body and thereby atrophying ghrelin cells by the oral intake of Orlistat over a minimum time period. Such methodology is represented in the drawings in FIGS. 1 through 8, wherein FIG. 1 represents a cross-sectional view of a normal human stomach before any Orlistat therapy; FIG. 2 represents a cross-sectional view of that normal human stomach shown in FIG. 1, after six months of Orlistat therapy, displaying a shrinkage thereof; FIG. 3 represents a cross-sectional view of that normal human stomach shown in FIG. 1 after twelve months of Orlistat therapy, displaying a further shrinkage thereof; FIG. 4 represents a cross-sectional view of normal human stomach in a “pre-prandial” or “pre-meal” state, with a circle placed thereon to designate a subsequently viewed enlarged portion thereof; FIG. 5 represents the enlarged portion shown within the drawing of FIG. 4, with large plump ghrelin cells, with abundant ghrelin hormone production released into the bloodstream from the ghrelin cells, the ghrelin circulating towards the hydrothalmus to stimulate an appetite; FIG. 6 represents a cross-sectional view of a pre-prandial stomach after six months of Orlistat therapy, with a circle placed thereon to designate a subsequently viewed enlarged portion thereof; with FIG. 7 representing the enlarged portion in the circle shown in FIG. 6 showing the grhelin cells smaller and partially atrophied, with some ghrelin cells completely atrophic, wherein they can not produce ghrelin, with much less ghrelin hormones shown in the blood vessels because of the atrophy of the ghrelin producing cells; with FIG. 8 representing a cross-sectional view of a pre-prandial stomach shown after twelve months of Orlistat therapy, and the Orlistat therapy has been discontinued after such a minimum inventive period, with a circle placed thereon to designate a subsequently viewed enlarged portion thereof; and wherein FIG. 9 representing the enlarged portion of FIG. 8 within the circle, showing ghrelin cells with some atrophied to a very small state, and most ghrelin cells completely atrophic, thereby now failing to produce ghrelin, and showing very little ghrelin hormones circulating in the bloodstream, thereby decreasing appetite, all of which comprises the etiology of reduced weight regain after such inventive prolonged Orlistat therapy.

The invention is thus a method of depressing serum ghrelin levels in a body by the step of: ingesting daily a compound of tetrahydrolipstatin (THL) for a period of at least one and preferably about two years. The invention includes a method of atrophying ghrelin levels in a human body for weight reduction and to permanently reduce appetite and eliminate weight regain by the step of: orally ingesting an admixture of tetrahydrolipstatin—THL on a regular basis for a period of at least eighteen months. The trahydrolipstatin comprises Orlistat. The invention also comprises a method of reducing post Orlistat therapy ghrelin production and eliminating associated weight regain by: maintaining an Orlistat therapy program for a period of at least about two years.

1. A method of depressing serum ghrelin levels in a body by the step of:
ingesting daily a compound of tetrahydrolipstatin (THL) for a period of at least two years.

2. A method of atrophying ghrelin levels in a human body for weight reduction and to permanently reduce appetite and eliminate weight regain by the step of:
orally ingesting an admixture of tetrahydrolipstatin—THL on a regular daily basis for a period of a minimum of eighteen months.

3. The method of claim 2, wherein said tetrahydrolipstatin comprises orlistat.

4. A method of reducing post Orlistat therapy ghrelin production and eliminating associated weight regain by:
maintaining a daily Orlistat therapy program for a period of at least two years said Orlistat taken at least once daily with a meal.
